(19) United States
(12) Patent Application Publication
(10) Pub. No.: US 2003/0091630 A1
(43) Pub. Date: May 15, 2003

(54) Formulation of an erodible, gastric retentive oral dosage form using in vitro disintegration test data

(76) Inventors: Jenny Louie-Helm, Union City, CA (US); Bret Berner, El Granada, CA (US)

Correspondence Address: REED & EBERLE LLP, 800 MENLO AVENUE, SUITE 210, MENLO PARK, CA, 94025, US

(21) Appl. No.: 10/014,750

(22) Filed: Oct. 25, 2001

(51) Int. Cl. A61K 9/22; A61K 9/14

(52) U.S. Cl. 424/468

(57) ABSTRACT

Erodible, gastric-retentive dosage forms are provided that are formulated using the in vitro drug release profile obtained with USP Disintegration test equipment rather the USP Dissolution Apparatus. The invention is premised on the discovery that the USP Disintegration Test and modified versions thereof are far more predictive of the in vivo release profile for a controlled release dosage form than is the standard USP Dissolution Test, particularly controlled release dosage forms of the swellable, erodible type. The dosage forms generally comprise particles of a biocompatible, hydrophilic polymer having the active agent incorporated therein, wherein the particles are optionally but preferably compacted into a tablet or loaded into a capsule. The dosage forms can be used to deliver water-insoluble or sparingly soluble drugs as well as water-soluble drugs, providing that the latter are coated with a protective coating or contained in a protective vesicle.

### TECHNICAL FIELD

The present invention relates generally to the field of drug delivery. More particularly, the invention relates to controlled release oral dosage forms formulated using in vitro data obtained using a disintegration test such as the established USP Disintegration Test, rather than the results obtained using a standard USP Dissolution Test, as is conventionally done.

### BACKGROUND OF THE INVENTION

Sustained release dosage forms for oral administration, designed to deliver a pharmacologically active agent over an extended time period, are well known. In particular, dosage forms that are capable of delivering drug to the stomach and gastrointestinal tract in a controlled, “sustained release” manner are described in U.S. Pat. Nos. 5,007,790 to Shell, 5,582,837 to Shell and 5,972,389 to Shell et al., all of common assignment herewith. The dosage forms described in the aforementioned patents are comprised of particles of a hydrophilic, water-swellable polymer with the drug dispersed therein. The polymeric particles in which the drug is dispersed absorb water, causing the particles to swell, which in turn promotes their retention in the stomach and also allows the drug contained in the particles to dissolve and then diffuse out of the particles. The polymeric particles also release drug as a result of physical erosion, i.e., degradation.

The aforementioned dosage forms are prepared based on the drug release profile obtained using the results of a standard in vitro USP Dissolution Test, as is conventionally done for controlled release dosage forms. See, for example, U.S. Pat. Nos. 6,093,420 to Baichwal; 6,143,322 to Sackler et al., 6,156,347 to Blatt et al.; 6,194,000 to Smith et al.; and 6,197,347 to Jan et al. That is, the components, relative quantities, and manufacturing processes are tailored to provide a particular release profile as modeled by a USP Dissolution Test, the assumption being that the standard USP Dissolution Test provides an accurate model for the drug release profile that will result in vivo, i.e., upon administration of a dosage form to a patient. Briefly, the standard USP Dissolution Test, as set forth in USP 24-NF 19, Supplement 4, Section 711, published by the United States Pharmacopeia & National Formulary in 2001, calls for immersion of a dosage in a specified solvent at 37° C. for a given time period, using either a basket stirring element or a paddle stirring element (respectively referred to as “Apparatus 1” and “Apparatus 2” in USP 24-NF 19). At regular time intervals, a sample of the solvent is withdrawn and the drug concentration therein determined. The USP Dissolution Test essentially represents the state of the art as a model for predicting the in vivo drug release profile of a controlled release dosage form.

For immediate release dosage forms, an additional test that is conventionally used to supplement dissolution as a predictor of the in vivo release profile is the USP Disintegration Test, described in USP 24-NF 19, supra, at Section 701. As explained therein, the test is not to be used for modified release dosage forms. The USP Disintegration Test is conducted by placing the dosage form to be tested in a basket-rack assembly, immersing the assembly in a specified fluid at a temperature between 35° C. and 39° C. for a given time period, and raising and lowering the basket in the immersion fluid through a distance of about 5.5 cm at a frequency of about 30 cycles per minute. The dosage forms are visually inspected at specified times for complete disintegration, defined in Section 701 of USP 24-NF 19 as the state in which any residue of the dosage form remaining in the basket rack of the test apparatus is a “soft mass having no palpably firm core.”

It has now been discovered, quite surprisingly, that the USP Disintegration Test, conducted for an extended time period, is a far more predictive test for drug release in vivo for controlled release dosage forms, particularly dosage forms of the swellable, erodible type to be administered with food as described in U.S. Pat. Nos. 5,007,790 to Shell, 5,582,837 to Shell and 5,972,389 to Shell et al., referenced above. To the best of applicants' knowledge, a controlled release dosage form formulated using the results of a USP Disintegration Test is completely new and unsuggested by the art.

### SUMMARY OF THE INVENTION

The present invention is directed to the aforementioned need in the art, and provides a method of formulating a controlled release dosage form, particularly of the swellable, erodible type, based on a desired in vitro profile obtained using a disintegration test, ideally the standard USP Disintegration Test, rather than a USP Dissolution Test. The method is premised on the discovery that the in vitro release profile of a controlled release dosage form obtained with a disintegration test is reliably predictive of the dosage form's actual drug release profile in vivo when administered with food (such that the stomach is in the “fed mode,” as will be described infra). The invention takes advantage of the correlation between the in vivo release profile and the in vitro release profile obtained using a disintegration test, wherein the correlation may be exact, linear, substantially linear, or otherwise predictable. With an exact correlation, the in vivo and in vitro release profiles will be the same, while with a linear or substantially linear correlation, the ratio of the in vivo disintegration rate to the disintegration rate obtained in vitro using a disintegration test is constant or substantially constant. After in vitro evaluation of candidate dosage forms (containing, for example, different components, or different quantities or types of the same components), a dosage form for in vivo use, i.e., for oral administration to a patient, is prepared based on the results obtained using the disintegration test.

The disintegration test used may be any suitable disintegration test that is predictive of drug release behavior in vivo, although a particularly preferred such test, as indicated above, is the standard USP Disintegration Test as set forth in USP 24-NF 19, Supplement 4, Section 701, published by the United States Pharmacopeia & National Formulary in 2001, or a modification of the standard test. The pertinent information obtained using the disintegration test is the “disintegration time,” a term that is used interchangeably herein with the terms “disintegration rate” and “in vitro release rate,” and refers to the time for complete disintegration of the dosage form to occur, wherein “complete disintegration” is as defined as the state in which less than 5% of the original dosage form remains visible.

The “disintegration time,” “release rate” and “release profile” in vivo refer to the time it takes for the orally administered dosage form (again, administered when the stomach is in the fed mode) to be reduced to 0-10% of its original size, as may be observed visually using NMR shift reagents or paramagnetic species, radio-opaque species or markers, or radiolabels. Unless otherwise indicated herein, all references to in vivo tests and in vivo results refer to results obtained upon oral administration of a dosage form with food, such that the stomach is in the fed mode.

The invention additionally provides controlled release dosage forms formulated using the aforementioned method. In one embodiment, a controlled release oral dosage form is provided for the continuous, controlled administration of a pharmacologically active agent to the stomach, duodenum and upper sections of the small intestine of a patient, the dosage form comprising a matrix having the active agent incorporated therein, wherein the matrix is comprised of a biocompatible, hydrophilic, erodible polymer that both swells in the presence of water and gradually erodes over a time period of hours—with swelling and erosion commencing upon contact with gastric fluid—and wherein the dosage form is formulated so as to provide an active agent release rate in vivo that correlates with the disintegration rate observed for the dosage form in vitro using a disintegration test. Generally, although not necessarily, drug release from the present dosage forms is erosion-controlled rather than swelling-controlled, although the initial swelling rate may initially be greater than the erosion rate; in the latter case, however, the erosion rate will generally surpass the swelling rate to deliver the full dose of the active agent. These dosage forms can minimize or even eliminate problems such as the overgrowth of detrimental intestinal flora resulting from drugs that are toxic to normal intestinal flora, by delivering the bulk of the drug dose to the upper G.I. tract and allowing little or no drug to reach the lower G.I. tract or colon. The dosage forms can also prevent chemical degradation of drugs by intestinal enzymes, as alluded to above, loss of bioavailability of a drug due to its leaving the acidic environment of the stomach, and chemical degradation of a drug in the neutral to alkaline environment of the gastrointestinal tract.

In another embodiment, an extended release oral dosage form is provided for 10 administering a pharmacologically active agent having little or no aqueous solubility (also referred to herein as “sparingly soluble drugs”) to the stomach and upper gastrointestinal tract of a patient, the dosage form comprising: a matrix comprised of a biocompatible, hydrophilic, erodible polymer that both swells in the presence of water and gradually erodes within the gastrointestinal (G.I.) tract; and, incorporated in the matrix, a pharmacologically active agent having an aqueous solubility of less than about 10 wt. % at 20° C., wherein the dosage form is formulated so as to provide an active agent release rate in vivo that corresponds to a desired active agent release profile obtained in vitro using a disintegration test.

While the dosage forms of the invention are primarily useful in conjunction with the delivery of sparingly soluble drugs, they may also be used to administer drugs having higher water solubility, i.e., active agents that may be quite soluble, or even completely soluble, in water. In this embodiment, the active agent may be blended with the polymer as with less soluble drugs or may be contained within a vesicle that prevents a too rapid release rate due to high drug solubility. Suitable vesicles include, but are not limited to, liposomes and nanoparticles, including nanocrystals, nanospheres and nanocapsules.

It has further been found that the rate of diffusion of the active agent out of the matrix can be slowed relative to the rate at which the active agent is released via polymer erosion by increasing drug particle size and selecting a polymer that will erode faster than it will swell.

In a further embodiment of this invention, the dosage form is a bilayer tablet with one layer comprised of a swellable polymer that erodes over a period longer than the drug delivery period and with the second layer containing drug and being erodible over the drug release period defined by the USP Disintegration Test. The function of the swelling layer is to provide sufficient particle size throughout the entire period of drug delivery to promote enable gastric retention in the fed mode.

The invention additionally provides a method for using these dosage forms to administer drugs on a continuous basis to the stomach, duodenum and upper sections of the small intestine. Dosage forms formulated so as to exhibit substantial swelling upon contact with gastrointestinal fluid provide for “gastric retention,” i.e., they are retained within the stomach for a period of hours if the fed mode has been induced. Such dosage forms are particularly useful for delivering drugs directly into the stomach for an extended period of time, and can therefore provide an effective means of treating local disorders of the stomach, e.g., Helicobacter pylori (“H. pylori”) infection, stomach ulcers, etc. The invention also encompasses a method for delivering drugs to the lower gastrointestinal tract, i.e., “below” the stomach, by administering a dosage form, as above, that is coated with an enteric coating material. The enteric coating material allows the dosage form to pass from the acidic environment of the stomach before they can dissolve and become available for absorption.

Details of these and other features of the invention will be apparent from the description that follows.

### BRIEF DESCRIPTION OF THE DRAWINGS

FIG. 1 is a graph comparing the percent of drug released from topiramate/polyethylene oxide dosage forms determined using a USP Disintegration Apparatus, the USP Dissolution Test, and in vivo, in beagle dogs, as described in Example 1.

FIG. 2 shows, in graph form, the release profile of a dosage form that was formulated to disintegrate in approximately 4 hours in a dog's stomach, and illustrates that the disintegration test was predictive of in vivo release, while the results of a USP Dissolution Test were not (see Example 1).

FIG. 3 is a graph comparing the extent of swelling for four controlled release, gastric-retentive (“GR”) dosage forms as evaluated in Example 2.

FIG. 4 illustrates the results of testing the four GR dosage forms using a USP Disintegration tester, as explained in Example 2.

FIG. 5 summarizes, in graph form, the erosion time of the four GR dosage forms in the stomach of dogs, evaluated in Example 2.

### DETAILED DESCRIPTION OF THE INVENTION

I. Definitions and Overview:

Before describing the present invention in detail, it is to be understood that this invention is not limited to specific active agents, dosage forms, dosing regimens, or the like, as such may vary. It is also to be understood that the terminology used herein is for the purpose of describing particular embodiments only, and is not intended to be limiting.

It must be noted that as used in this specification and the appended claims, the singular forms “a,” “an” and “the” include plural referents unless the context clearly dictates otherwise. Thus, for example, reference to “an active agent” or “a pharmacologically active agent” includes a single active agent as well a two or more different active agents in combination, reference to “a polymer” includes mixtures of two or more polymers as well as a single polymer, and the like.

In describing and claiming the present invention, the following terminology will be used in accordance with the definitions set out below.

The terms “drug,” “active agent,” and “pharmacologically active agent” are used interchangeably herein to refer to any chemical compound, complex or composition that is suitable for oral administration and that has a beneficial biological effect, preferably a therapeutic effect in the treatment of a disease or abnormal physiological condition. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent,” “pharmacologically active agent” and “drug” are used, then, or when a particular active agent is specifically identified, it is to be understood that applicants intend to include the active agent per se as well as pharmaceutically acceptable, pharmacologically active salts, esters, amides, prodrugs, metabolites, analogs, etc.

The term “dosage form” denotes any form of a pharmaceutical composition that contains an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with: (1) the characteristics of the particular drug, including both its pharmacological characteristics and its physical characteristics, such as solubility; (2) the characteristics of the swellable matrix, such as its permeability; and (3) the relative amounts of the drug and polymer. In most cases, the dosage form will be such that effective results will be achieved with administration no more frequently than once every eight hours or more, preferably once every twelve hours or more, and even more preferably once every twenty-four hours or more.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual by inhibiting or causing regression of a disorder or disease.

By an “effective” amount or a “therapeutically effective amount” of a drug or pharmacologically active agent is meant a nontoxic but sufficient amount of the drug or agent to provide the desired effect.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative, refers to a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term, “pharmaceutically acceptable” is used to refer to an excipient, it implies that the excipient has met the required standards of toxicological and manufacturing testing or that it is on the Inactive Ingredient Guide prepared by the FDA.

The term “biocompatible” is used interchangeably with the term “pharmaceutically acceptable.”

The term “soluble”, as used herein, refers to a drug having a solubility (measured in water at 20° C.) in the range of 2% to greater than 50% by weight, more preferably 10% to greater than 40% by weight. The terms “sparingly soluble” and “slightly soluble” refer to a drug having a solubility (measured in water at 20° C.) in the range of 0.001% to about 5% by weight, more preferably 0.001% to 3% by weight. Such drugs are also referred to as having “low” or “poor” aqueous solubility.

The term “vesicle,” as used herein, refers to a small (usually 0.01 to 1.0 mm), usually spherical, membrane-bound structure that may contain or be composed of either lipoidal or aqueous material, or both. Suitable vesicles include, but are not limited to, liposomes, nanoparticles, and microspheres composed of amino acids. While some of these particles, especially nanoparticles and microspheres, need not be membrane-bound structures, for the purposes of the present invention, they are encompassed by the term “vesicle.”

The term “controlled release” is intended to refer to any drug-containing formulation in which release of the drug is not immediate, i.e., with a “controlled release” formulation, oral administration does not result in immediate release of the drug into an absorption pool. The term is used interchangeably with “nonimmediate release” as defined in Remington: The Science and Practice of Pharmacy, Nineteenth Ed. (Easton, Pa.: Mack Publishing Company, 1995). As discussed therein, immediate and nonimmediate release can be defined kinetically by reference to the following equation:

$$\begin{matrix}Dosage \\ Form\end{matrix} {{\to }_{\begin{matrix}drug \\ release \end{matrix}}}^{ {k}_{r} } \begin{matrix}Absorption \\ Pool\end{matrix} {{\to }_{\begin{matrix} absorption \\ \end{matrix}}}^{ {k}_{a} } \begin{matrix}Target \\ Area\end{matrix} {{\to }_{\begin{matrix} elimination \\ \end{matrix}}}^{ {k}_{e} }$$

The “absorption pool” represents a solution of the drug administered at a particular absorption site, and kᵣ, kₐ and kₑ are first-order rate constants for (1) release of the drug from the formulation, (2) absorption, and (3) elimination, respectively. For immediate release dosage forms, the rate constant for drug release kᵣ is far greater than the absorption rate constant kₐ. For controlled release formulations, the opposite is true, i.e., kᵣ<<kₐ, such that the rate of release of drug from the dosage form is the rate-limiting step in the delivery of the drug to the target area. It should be noted that this simplified model uses a single first order rate constant for release and absorption, and that the controlled release kinetics with any particular dosage form may be much for complicated. In general, however, the term “controlled release” as used herein includes any nonimmediate release formulation, including but not limited to sustained release, delayed release and pulsatile release formulations.

The term “sustained release” is used in its conventional sense to refer to a drug formulation that provides for gradual release of a drug over an extended period of time, and that preferably, although not necessarily, results in substantially constant blood levels of a drug over an extended time period.

The terms “hydrophilic” and “hydrophobic” are generally defined in terms of a partition coefficient P, which is the ratio of the equilibrium concentration of a compound in an organic phase to that in an aqueous phase. A hydrophilic compound has a P value less than 1.0, typically less than about 0.5, where P is the partition coefficient of the compound between octanol and water, while hydrophobic compounds will generally have a P greater than about 1.0, typically greater than about 5.0. The polymeric carriers herein are hydrophilic, and thus compatible with aqueous fluids such as those present in the human body.

The term “polymer” as used herein refers to a molecule containing a plurality of covalently attached monomer units, and includes branched, dendrimeric and star polymers as well as linear polymers. The term also includes both homopolymers and copolymers, e.g., random copolymers, block copolymers and graft copolymers, as well as uncrosslinked polymers and slightly to moderately to substantially crosslinked polymers.

The terms “swellable” and “bioerodible” (or simply “erodible”) are used to refer to the preferred polymers herein, with “swellable” polymers being those that are capable of absorbing water and physically swelling as a result, with the extent to which a polymer can swell being determined by the degree of crosslinking, and “bioerodible” or “erodible” polymers referring to polymers that slowly dissolve and/or gradually hydrolyze in an aqueous fluid, and/or that physically erodes as a result of movement within the stomach or gastrointestinal tract.

The term “fed mode,” as used herein, refers to a state which is typically induced in a patient by the presence of food in the stomach, the food giving rise to two signals, one that is said to stem from stomach distension and the other a chemical signal based on food in the stomach. It has been determined that once the fed mode has been induced, larger particles are retained in the stomach for a longer period of time than smaller particles. Thus, the fed mode is typically induced in a patient by the presence of food in the stomach.

In the normal digestive process, the passage of matter through the stomach is delayed by a physiological condition that is variously referred to as the digestive mode, the postprandial mode, or the “fed mode.” Between fed modes, the stomach is in the interdigestive or “fasting” mode. The difference between the two modes lies in the pattern of gastroduodenal motor activity.

In the fasting mode, the stomach exhibits a cyclic activity called the interdigestive migrating motor complex (“IMMC”). This activity occurs in four phases:

Phase I, which lasts 45 to 60 minutes, is the most quiescent, with the stomach experiencing few or no contractions;

Phase II, characterized by sweeping contractions occurring in an irregular intermittent pattern and gradually increasing in magnitude;

Phase III, consisting of intense bursts of peristaltic waves in both the stomach and the small bowel, lasting for about 5 to 15 minutes; and

Phase IV is a transition period of decreasing activity which lasts until the next cycle begins.

The total cycle time for all four phases is approximately 90 minutes. The greatest activity occurs in Phase III, when powerful peristaltic waves sweep the swallowed saliva, gastric secretions, food particles, and particulate debris, out of the stomach and into the small intestine and colon. Phase III thus serves as an intestinal housekeeper, preparing the upper tract for the next meal and preventing bacterial overgrowth.

The fed mode is initiated by nutritive materials entering the stomach upon the ingestion of food. Initiation is accompanied by a rapid and profound change in the motor pattern of the upper gastrointestinal tract, over a period of 30 seconds to one minute. The change is observed almost simultaneously at all sites along the G.I. tract and occurs before the stomach contents have reached the distal small intestine. Once the fed mode is established, the stomach generates 3-4 continuous and regular contractions per minute, similar to those of the fasting mode but with about half the amplitude. The pylorus is partially open, causing a sieving effect in which liquids and small particles flow continuously from the stomach into the intestine while indigestible particles greater in size than the pyloric opening are retropelled and retained in the stomach. This sieving effect thus causes the stomach to retain particles exceeding about 1 cm in size for approximately 4 to 6 hours.

In one embodiment of the invention, the present drug delivery systems are used to administer a drug of limited aqueous solubility. That is, the transit time through the gastrointestinal tract often limits the amount of drug available for absorption at its most efficient absorption site, or for local activity at one segment of the G.I. tract. The latter is particularly true when the absorption site, or site of local action, is high in the G.I. tract, for example, when the required treatment is local in the stomach as is often the case with ulcers. As the solubility of the drug decreases, the time required for drug dissolution and absorption through the intestinal membrane becomes less adequate and, thus, the transit time becomes a significant factor that interferes with effective drug delivery. To counter this, oral administration of sparingly soluble drugs is done frequently, often several times per day. Moreover, due to their insolubility, sparingly soluble or almost insoluble drugs cannot readily be delivered by either solution-diffusion or membrane-controlled delivery systems. The present dosage forms, like the dosage forms of the aforementioned '389 patent, provide for effective delivery of sparingly soluble drugs. In contrast to the dosage forms of the '389 patent, however, the composition of the present dosage forms is determined by using the results of a USP Disintegration Test, discussed infra, rather than the USP Dissolution Test, and thus a desired drug release profile that reflects in vivo drug absorption can be obtained with greater accuracy.

In a related embodiment, the drug delivery systems are used to administer a drug of unspecified solubility in water. In this case, however, the drug particles of the dosage forms are either encased in protective vesicles such as liposomes or the like, and/or coated, typically with an enteric coating.

In a further embodiment of this invention, the dosage form is a bilayer tablet having a first layer comprised of a swellable polymer that erodes over a period longer than the drug delivery period, and a second layer containing drug and being erodible over a drug release period that is predicted using a USP Disintegration Test as will be discussed in detail infra. The function of the swelling layer is to provide sufficient particle size throughout the entire period of drug delivery to enable gastric retention in the fed mode.

Accordingly, the dosage forms of the invention are comprised of at least one biocompatible, hydrophilic, erodible polymer with a drug dispersed therein, wherein the composition of the dosage form is optimized using standard USP disintegration test equipment. The swelling properties of the polymers can be important in that they allow the dosage forms to be retained in the stomach where they effectively deliver drugs on a continuous basis to the stomach, duodenum and upper sections of the small intestine where absorption is efficient. For drug delivery to the stomach, a polymer is used that (i) swells unrestrained dimensionally via imbibition of gastric fluid to increase the size of the particles to promote gastric retention within the stomach of a patient in which the fed mode has been induced, (ii) gradually erodes over a time period of hours, with the erosion commencing upon contact with the gastric fluid, and (iii) releases the drug to the stomach and duodenum at a rate dependent on the erosion rate. Preferred dosage forms have an erosion rate that is faster than the swelling rate, i.e., drug release from the dosage form is primarily controlled by polymer erosion than by polymer swelling.

II. Dosage Form Optimization Using a Disintegration Test:

The preferred composition of a dosage form of the invention, i.e., a dosage form that will give rise to a desired drug release profile in vivo, is determined experimentally, in vitro, using a suitable disintegration test. That is, one or more matrix polymers are selected along with an active agent to be administered, and different dosage forms are prepared using different matrix polymers and/or active agents, matrix polymers of different molecular weights, matrix polymers crosslinked to different degrees, and/or different amounts of the different components. The pertinent information obtained using the disintegration test is the “disintegration time,” a term that is used interchangeably herein with the terms “disintegration rate” and “in vitro release rate,” and refers to the time for complete disintegration of the dosage form to occur, wherein “complete disintegration” is as defined as less than 5% of the dosage form (or 5% of the active agent-containing layer in a bilayer or trilayer tablet) remaining visible. If the test is stopped prior to complete disintegration, the fraction of the dosage form remaining is noted along with the time of the monitoring period. The “disintegration time,” “release rate” and “release profile” in vivo refer to the time it takes for the orally administered dosage form (again, administered when the stomach is in the fed mode) to be reduced to 0-10% of its original size, as may be observed visually using NMR shift reagents or paramagnetic species, radio-opaque species or objects, or radiolabels. Preferably, the present dosage forms release at least 75 wt. % of the active agent, more preferably at least 85 wt. % of the active agent, during gradual erosion of the dosage forms in the stomach and gastrointestinal tract.

The USP Disintegration Test, used in conjunction with the disintegration test equipment described in USP 24-NF 19, supra, at Section 701, is a preferred disintegration test. As explained in the aforementioned section of USP 24-NF 19, the apparatus consists of a basket-rack assembly, a 1000-ml beaker, 142 to 148 mm in height and having an outside diameter of 103 to 108 mm, a thermostatic arrangement for heating an immersion fluid between 35° C. and 39° C., and a device for raising and lowering the basket in the immersion fluid at a constant frequency rate between 29 and 32 cycles per minute through a distance of 5.3 cm to 5.7 cm. The time required for the upward and downward strokes is the same, and the volume of the fluid in the vessel is such that the wire mesh of the basket remains at least 2.5 cm below the fluid surface on the upward stroke, and should not descend to within less than 2.5 cm of the bottom of the vessel on the downward stroke. There should be no appreciable horizontal movement of the basket rack assembly; the assembly moves solely in a vertical direction, along its axis. The basket-rack assembly consists of six open-ended transparent tubes, each having dimensions specified in the aforementioned section of USP 24-NF 19; the tubes are held in a vertical position by two plastic plates, with six holes equidistance from the center of the plate and equally spaced from one another. Attached to the undersurface of the lower plate is a woven stainless steel wire mesh. A suitable means is provided to suspend the basket-rack assembly from a raising and lowering device.

Accordingly, the standard USP Disintegration Test is conducted using the above-described test equipment by placing the dosage form to be tested in each basket-rack assembly, immersing the assembly in a specified fluid at a temperature between 35° C. and 39° C. for a given time period, and raising and lowering the basket in the immersion fluid through a distance of about 5.5 cm at a frequency of about 30 cycles per minute. The dosage forms are visually inspected at specified times for complete disintegration. The particularly preferred disintegration test used in conjunction with the invention is a modification of the standard USP Disintegration Test wherein an extended monitoring time is used, e.g., a four- to eight-hour time period, and wherein a thin plastic disk (9.5±0.15 mm in thickness, 20.7±0.15 mm in diameter) is placed on each dosage form (noted as optional in Section 701 of USP 24-NF 19).

To use the aforementioned disintegration test as a predictor of in vivo drug release from the controlled release dosage forms described herein, a correlation should be first established between the release profile of a particular dosage form obtained using an in vitro disintegration as just described and the release profile of that dosage form obtained in vivo, using animal test subjects. It will be seen that there is a correlation between the release profile obtained using an in vitro disintegration test and the release profile obtained in vivo, enabling the in vitro test to be used as predictive of in vivo behavior (see Examples 1 and 2). The correlation may be exact, or it may be linear or substantially linear.

Once the correlation between the in vitro disintegration test results and in vivo behavior has been established for a particular dosage form, a plurality of different candidate dosage forms is prepared, with each dosage form comprised of a biocompatible, hydrophilic polymer and a pharmacologically active agent incorporated therein. As noted above, the dosage forms may contain different polymers, compositionally identical polymers having different molecular weights or different degrees of crosslinking, etc. Then, the in vitro drug release profile is obtained for each candidate dosage form in an aqueous medium in a USP disintegration tester using the same test that was employed in determining the correlation between the in vitro and in vivo tests as described above. The in vitro drug release profiles obtained are then analyzed, and a determination is made as to which of the in vitro drug release profiles corresponds most closely to a desired in vivo drug release profile. The dosage form having the determined in vitro drug release profile is then selected for administration to a patient.

II. Swellable, Bioerodible Polymers:

With the present dosage forms, the rate at which the drug is released to the gastrointestinal tract is largely dependent on the rate at which the polymer matrix erodes and on the degree to which the polymer swells. The polymer used in the dosage forms of the present invention should not release the drug at too rapid a rate so as to result in a drug overdose or rapid passage into and through the gastrointestinal tract (i.e., in less than about four hours), nor should the polymer release drug too slowly to achieve the desired biological effect. Thus, polymers that permit a rate of drug release that achieves the requisite pharmacokinetics for a desired duration, as determined using a USP Disintegration Test, are selected for use in the dosage forms of the present invention.

Polymers suitable for use in the present invention are those that both swell upon absorption of gastric fluid and gradually erode over a time period of hours. Erosion initiates simultaneously with the swelling process, upon contact of the surface of the dosage form with gastric fluid. Erosion reflects the dissolution of the polymer beyond the polymer gel-solution interface where the polymer has become sufficiently dilute that it can be transported away from the dosage form by diffusion or convection. This may also depend on the hydrodynamic and mechanical forces present in the gastrointestinal tract during the digestive process. While swelling and erosion occur at the same time, it is preferred herein that drug release should be erosion-controlled, meaning that the selected polymer should be such that complete drug release occurs primarily as a result of erosion rather than swelling and dissolution. However, swelling should take place at a rate that is sufficiently fast to allow the tablet to be retained in the stomach. At minimum, for an erosional gastric retentive dosage form, there should be an extended period during which the dosage form maintains its size before it is diminished by erosion.

Suitable polymers for use in the present dosage forms may be linear, branched, dendrimeric, or star polymers, and include synthetic hydrophilic polymers as well as semi-synthetic and naturally occurring hydrophilic polymers. The polymers may be homopolymers or copolymers, if copolymers, either random copolymers, block copolymers or graft copolymers. Synthetic hydrophilic polymers useful herein include, but are not limited to:

polyalkylene oxides, particularly poly(ethylene oxide), polyethylene glycol and poly(ethylene oxide)-poly(propylene oxide)copolymers;

cellulosic polymers;

acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate;

maleic anhydride copolymers;

polymaleic acid;

poly(acrylamides) such as polyacrylamide per se, poly(methacrylamide), poly(dimethylacrylamide), and poly(N-isopropyl-acrylamide);

poly(olefinic alcohol)s such as poly(vinyl alcohol);

poly(N-vinyl lactams) such as poly(vinyl pyrrolidone), poly(N-vinyl caprolactam), and copolymers thereof;

polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimethylene glycol;

polyoxyethylated sorbitol and polyoxyethylated glucose;

polyoxazolines, including poly(methyloxazoline) and poly(ethyloxazoline);

polyvinylamines;

polyvinylacetates, including polyvinylacetate per se as well as ethylene-vinyl acetate copolymers, polyvinyl acetate phthalate, and the like;

polyimines, such as polyethyleneimine;

starch and starch-based polymers;

polyurethane hydrogels;

chitosan;

polysaccharide gums;

zein; and

shellac, ammoniated shellac, shellac-acetyl alcohol, and shellac n-butyl stearate.

The term “cellulosic polymer” is used herein to denote a linear polymer of anhydroglucose. Cellulosic polymers that can be used advantageously in the present dosage forms include, without limitation, hydroxymethylcellulose, hydroxypropylcellulose, hydroxyethylcellulose, hydroxypropyl methylcellulose, methylcellulose, ethylcellulose, cellulose acetate, cellulose acetate phthalate, cellulose acetate trimellitate, hydroxypropyl methylcellulose phthalate, hydroxypropylcellulose phthalate, cellulose hexahydrophthalate, cellulose acetate hexahydrophthalate, carboxymethylcellulose, carboxymethylcellulose sodium, and microcrystalline cellulose. Preferred cellulosic polymers are alkyl-substituted cellulosic polymers that ultimately dissolve in the GI tract in a predictably delayed manner. Preferred alkyl-substituted cellulose derivatives are those substituted with alkyl groups of 1 to 3 carbon atoms each. Examples are methylcellulose, hydroxymethylcellulose, hydroxyethylcellulose, hydroxypropylcellulose, hydroxypropyl methylcellulose, and carboxymethylcellulose. In terms of their viscosities, one class of preferred alkyl-substituted celluloses includes those whose viscosity is within the range of about 50 to about 110,000 centipoise as a 2% aqueous solution at 20° C. Another class includes those whose viscosity is within the range of about 800 to about 6,000 centipoise as a 1% aqueous solution at 20° C. Particularly preferred alkyl-substituted celluloses are hydroxyethylcellulose and hydroxypropylmethylcellulose. A presently preferred hydroxyethylcellulose is NATRASOL® 250HX NF (National Formulary), available from Aqualon Company, Wilmington, Del., USA.

Polyalkylene oxides are the preferred polymers herein, and the polyalkylene oxides that are of greatest utility are those having the properties described above for alkyl-substituted cellulose polymers. A particularly preferred polyalkylene oxide is poly(ethylene oxide), which term is used herein to denote a linear polymer of unsubstituted ethylene oxide. Poly(ethylene oxide)s are often characterized by their viscosity in solution. For purposes of this invention, a preferred viscosity range is about 50 to about 2,000,000 centipoise for a 2% aqueous solution at 20° C. Preferred poly(ethylene oxide)s are Polyox® 303, Polyox® Coag, Polyox® 301, Polyox® WSR N-60K, Polyox® WSR 1105 and Polyox® WSR N-80, having number average molecular weights of 7 million, 5 million, 4 million, 2 million, 900,000 and 200,000, respectively, all products of Union Carbide Chemicals and Plastics Company Inc. of Danbury, Conn., USA.

Polysaccharide gums, both natural and modified (semi-synthetic) can be used. Examples are dextran, xanthan gum, gellan gum, welan gum and rhamsan gum. Xanthan gum is preferred.

Crosslinked polyacrylic acids of greatest utility are those whose properties are the same as those described above for alkyl-substituted cellulose and polyalkylene oxide polymers. Preferred crosslinked polyacrylic acids are those with a viscosity ranging from about 4,000 to about 40,000 centipoise for a 1% aqueous solution at 25° C. Three presently preferred examples are CARBOPOL® NF grades 971P, 974P and 934P (BF Goodrich Co., Specialty Polymers and Chemicals Div., Cleveland, Ohio, USA). Further examples are polymers known as WATER LOCK®, which are starch/acrylates/acrylamide copolymers available from Grain Processing Corporation, Muscatine, Iowa, USA.

Suitable polymers also include naturally occurring hydrophilic polymers such as, by way of example, proteins such as collagen, fibronectin, albumins, globulins, fibrinogen, fibrin and thrombin; aminated polysaccharides, particularly the glycosaminoglycans, e.g., hyaluronic acid, chitin, chondroitin sulfate A, B, or C, keratin sulfate, keratosulfate and heparin; guar gum; xanthan gum; carageenan; alginates; pectin; and activated polysaccharides such as dextran and starches.

The aforementioned list of polymers is not exhaustive, and a variety of other synthetic hydrophilic polymers may be used, as will be appreciated by those skilled in the art.

The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water. Biodegradable segments may be composed of small molecular segments such as ester linkages, anhydride linkages, ortho ester linkages, ortho carbonate linkages, amide linkages, phosphonate linkages, etc.

Any polymer or polymers of the matrix may also be crosslinked, with the degree of crosslinking directly affecting the rate of polymer swelling as well as the erosion rate. That is, a polymer having a higher degree of crosslinking will exhibit less swelling and slower erosion than a polymer having a lower degree of crosslinking. Crosslinked polymers may be prepared using the above-mentioned exemplary polymers using conventional crosslinking procedures (e.g., chemical crosslinking with an added crosslinking agent, photolytically induced crosslinking, etc.), or the polymers may be obtained commercially in crosslinked form.

The water-swellable polymers can be used individually or in combination. Certain combinations will often provide a more controlled release of the drug than their components when used individually. Examples include, but are not limited to, the following: a cellulosic polymer combined with a gum, such as hydroxyethylcellulose or hydroxypropylcellulose combined with xanthan gum; a polyalkylene oxide combined with a gum, such as poly(ethylene oxide) combined with xanthan gum; and a polyalkylene oxide combined with a cellulosic polymer, such as poly(ethylene oxide) combined with hydroxyethylcellulose or hydroxypropylcellulose.

Combinations of different poly(ethylene oxide)s are also contemplated, with polymers of different molecular weights contributing to different dosage form characteristics. For example, a very high molecular weight poly(ethylene oxide) such as Polyox® 303 (with a number average molecular weight of 7 million) or Polyox® Coag (with a number average molecular weight of 5 million) may be used to significantly enhance diffusion relative to disintegration release by providing high swelling as well as tablet integrity. Incorporating a lower molecular weight poly(ethylene oxide) such as Polyox® WSR N-60K (number average molecular weight approximately 2 million) with Polyox® 303 and/or Polyox® Coag increases disintegration rate relative to diffusion rate, as the lower molecular weight polymer reduces swelling and acts as an effective tablet disintegrant. Incorporating an even lower molecular weight poly(ethylene oxide) such as Polyox® WSR N-80 (number average molecular weight approximately 200,000) further increases disintegration rate.

The hydrophilicity and water swellability of these polymers cause the drug-containing matrices to swell in size in the gastric cavity due to ingress of water in order to achieve a size that will be retained in the stomach when introduced during the fed mode. These qualities also cause the matrices to become slippery, which provides resistance to peristalsis and further promotes their retention in the stomach. The release rate of a drug from the matrix is primarily dependent upon the rate of water imbibition and the rate at which the drug dissolves and diffuses from the swollen polymer, which in turn is related to the solubility and dissolution rate of the drug, the drug particle size and the drug concentration in the matrix.

The amount of polymer relative to the drug can vary, depending on the drug release rate desired and on the polymer, its molecular weight, and excipients that may be present in the formulation. The amount of polymer will be sufficient however to retain at least about 40% of the drug within the matrix one hour after ingestion (or immersion in the gastric fluid). Preferably, the amount of polymer is such that at least 50% of the drug remains in the matrix one hour after ingestion. More preferably, at least 60%, and most preferably at least 80%, of the drug remains in the matrix one hour after ingestion. In all cases, however, substantially all of the drug will be released from the matrix within about eight hours, and preferably within about six hours, after ingestion, “substantially all” meaning at least 85%, preferably at least 90%.

It has now been found that higher molecular weight polymers are preferred to provide a desired extended release profile using the present dosage forms. Suitable molecular weights are generally in the range of about 5,000 to about 20,000,000. For sparingly soluble drugs, the polymers have molecular weights preferably in the range of about 5,000 to about 8,000,000, more preferably in the range of about 10,000 to about 5,000,000. For water-soluble drugs, the polymers preferably have molecular weights of at least about 10,000, but the molecular weight used will vary with the selected polymer. For example, for hydroxypropyl methylcellulose, the minimum molecular weight may be as low as 10,000, while for poly(ethylene oxide)s the molecular weight may be far higher, on the order of 2,000,000 or more.

IV. Active Agents

The dosage forms of the present invention are effective for the continuous, controlled administration of drugs that are capable of acting either locally within the gastrointestinal tract, or systemically by absorption into circulation via the gastrointestinal mucosa. Gastric-retentive dosage forms such as those disclosed and claimed herein are particularly useful for the delivery of drugs that are relatively insoluble, are ionized within the gastrointestinal tract, or require active transport.

The active agent administered may be any compound that is suitable for oral drug administration; examples of the various classes of active agents that can be administered using the present dosage forms include, but are not limited to: analgesic agents; anesthetic agents; antiarthritic agents; respiratory drugs; anticancer agents; anticholinergics; anticonvulsants; antidepressants; antidiabetic agents; antidiarrheals; antihelminthics; antihistamines; antihyperlipidemic agents; antihypertensive agents; anti-infective agents such as antibiotics and antiviral agents; antiinflammatory agents; antimigraine preparations; antinauseants; antineoplastic agents; antiparkinsonism drugs; antipruritics; antipsychotics; antipyretics; antispasmodics; antitubercular agents; antiulcer agents and other gastrointestinally active agents; antiviral agents; anxiolytics; appetite suppressants; attention deficit disorder (ADD) and attention deficit hyperactivity disorder (ADHD) drugs; cardiovascular preparations including calcium channel blockers, CNS agents, and vasodilators; beta-blockers and antiarrhythmic agents; central nervous system stimulants; cough and cold preparations, including decongestants; diuretics; genetic materials; herbal remedies; hormonolytics; hypnotics; hypoglycemic agents; immunosuppressive agents; leukotriene inhibitors; mitotic inhibitors; muscle relaxants; narcotic antagonists; nutritional agents, such as vitamins, essential amino acids and fatty acids; parasympatholytics; peptide drugs; psychostimulants; sedatives; steroids; sympathomimetics; and tranquilizers.

Commonly known drugs that are water insoluble or are sparingly soluble in water include, by way of example, the following:

Gastrointestinally active agents. Gastrointestinally active agents are particularly preferred drugs that can be administered using the present dosage forms These types of drugs include agents for inhibiting gastric acid secretion, such as the H₂ receptor antagonists cimetidine, ranitidine, famotidine, and nizatidine, the H⁺, K⁺-ATPase inhibitors (also referred to as “proton pump inhibitors”) omeprazole and lansoprazole, and antacids such as calcium carbonate, aluminum hydroxide, and magnesium hydroxide. Also included within this general group are agents for treating infection with Helicobacter pylori (H. pylori), such as metronidazole, tinidazole, amoxicillin, clarithromycin, tetracycline, thiamphenicol, and bismuth compounds (e.g., bismuth subcitrate and bismuth subsalicylate). Other gastrointestinally active agents administrable using the present dosage forms include, but are not limited to, pentagastrin, carbenoxolone, sulfated polysaccharides such as sucralfate, prostaglandins such as misoprostol, and muscarinic antagonists such as pirenzepine and telenzepine. Additionally included are antidiarrheal agents, antiemetic agents and prokinetic agents such as ondansetron, granisetron, metoclopramide, chlorpromazine, perphenazine, prochlorperazine, promethazine, thiethylperazine, triflupromazine, domperidone, trimethobenzamide, cisapride, motilin, loperamide, diphenoxylate, and octreotide.

Anti-microbial agents. These include: tetracycline antibiotics and related compounds (chlortetracycline, oxytetracycline, demeclocycline, methacycline, doxycycline, minocycline, rolitetracycline); macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g., cloxacillin, dicloxacillin, nafcillin, and oxacillin), extended spectrum penicillins (e.g., aminopenicillins such as ampicillin and amoxicillin, and the antipseudomonal penicillins such as carbenicillin), and cephalosporins (e.g., cefadroxil, cefepime, cephalexin, cefazolin, cefoxitin, cefotetan, cefuroxime, cefotaxime, ceftazidime, and ceftriaxone), and carbapenems such as imipenem, meropenem and aztreonam; aminoglycoside antibiotics such as streptomycin, gentamicin, tobramycin, amikacin, and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole, and sulfamethoxazole; quinolone antibiotics such as ciprofloxacin, nalidixic acid, and ofloxacin; anti-mycobacterials such as isoniazid, rifampin, rifabutin, ethambutol, pyrazinamide, ethionamide, aminosalicylic, and cycloserine; systemic antifungal agents such as itraconazole, ketoconazole, fluconazole, and amphotericin B; antiviral agents such as acyclovir, famcicylovir, ganciclovir, idoxuridine, sorivudine, trifluridine, valacyclovir, vidarabine, didanosine, stavudine, zalcitabine, zidovudine, amantadine, interferon alpha, ribavirin and rimantadine; and miscellaneous antimicrobial agents such as chloramphenicol, spectinomycin, polymyxin B (colistin), bacitracin, nitrofurantoin, methenamine mandelate and methenamine hippurate.

Anti-diabetic agents. These include, by way of example, acetohexamide, chlorpropamide, ciglitazone, gliclazide, glipizide, glucagon, glyburide, miglitol, pioglitazone, tolazamide, tolbutamide, triampterine, and troglitazone.

Analgesics. Non-opioid analgesic agents include apazone, etodolac, difenpiramide, indomethacin, meclofenamate, mefenamic acid, oxaprozin, phenylbutazone, piroxicam, and tolmetin; opioid analgesics include alfentanil, buprenorphine, butorphanol, codeine, drocode, fentanyl, hydrocodone, hydromorphone, levorphanol, meperidine, methadone, morphine, nalbuphine, oxycodone, oxymorphone, pentazocine, propoxyphene, sufentanil, and tramadol.

Anti-inflammatory agents. Anti-inflammatory agents include the nonsteroidal anti-inflammatory agents, e.g., the propionic acid derivatives as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, and fenbufen; apazone; diclofenac; difenpiramide; diflunisal; etodolac; indomethacin; ketorolac; meclofenamate; nabumetone; phenylbutazone; piroxicam; sulindac; and tolmetin. Steroidal anti-inflammatory agents include hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, and methylprednisolone.

Anti-convulsant agents. Suitable anti-convulsant (anti-seizure) drugs include, by way of example, azetazolamide, carbamazepine, clonazepam, clorazepate, ethosuximide, ethotoin, felbamate, lamotrigine, mephenytoin, mephobarbital, phenytoin, phenobarbital, primidone, trimethadione, vigabatrin, topiramate, and the benzodiazepines. Benzodiazepines, as is well known, are useful for a number of indications, including anxiety, insomnia, and nausea.

CNS and respiratory stimulants. CNS and respiratory stimulants also encompass a number of active agents. These stimulants include, but are not limited to, the following: xanthines such as caffeine and theophylline; amphetamines such as amphetamine, benzphetamine hydrochloride, dextroamphetamine, dextroamphetamine sulfate, levamphetamine, levamphetamine hydrochloride, methamphetamine, and methamphetamine hydrochloride; and miscellaneous stimulants such as methylphenidate, methylphenidate hydrochloride, modafinil, pemoline, sibutramine, and sibutramine hydrochloride.

Neuroleptic agents. Neuroleptic drugs include antidepressant drugs, antimanic drugs, and antipsychotic agents, wherein antidepressant drugs include (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors citalopram, fluoxetine, fluvoxamine, paroxetine, sertraline, and venlafaxine, (c) monoamine oxidase inhibitors such as phenelzine, tranylcypromine, and (−)-selegiline, and (d) other, “atypical” antidepressants such as nefazodone, trazodone and venlafaxine, and wherein antimanic and antipsychotic agents include (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

Hypnotic agents and sedatives include clomethiazole, ethinamate, etomidate, glutethimide, meprobamate, methyprylon, zolpidem, and barbiturates (e.g., amobarbital, apropbarbital, butabarbital, butalbital, mephobarbital, methohexital, pentobarbital, phenobarbital, secobarbital, thiopental).

Anxiolytics and tranquilizers include benzodiazepines (e.g., alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, oxazepam, prazepam, quazepam, temazepam, triazolam), buspirone, chlordiazepoxide, and droperidol.

Anticancer agents, including antineoplastic agents: Paclitaxel, docetaxel, camptothecin and its analogues and derivatives (e.g., 9-aminocamptothecin, 9-nitrocamptothecin, 10-hydroxy-camptothecin, irinotecan, topotecan, 20-O-β-glucopyranosyl camptothecin), taxanes (baccatins, cephalomannine and their derivatives), carboplatin, cisplatin, interferon-α_{2A}, interferon-α_{2B}, interferon-α_{N3} and other agents of the interferon family, levamisole, altretamine, cladribine, tretinoin, procarbazine, dacarbazine, gemcitabine, mitotane, asparaginase, porfimer, mesna, amifostine, mitotic inhibitors including podophyllotoxin derivatives such as teniposide and etoposide and vinca alkaloids such as vinorelbine, vincristine and vinblastine.

Antihyperlipidemic agents. Lipid-lowering agents, or “hyperlipidemic” agents,” include HMG-CoA reductase inhibitors such as atorvastatin, simvastatin, pravastatin, lovastatin and cerivastatin, and other lipid-lowering agents such as clofibrate, fenofibrate, gemfibrozil and tacrine.

Anti-hypertensive agents. These include amlodipine, benazepril, darodipine, dilitazem, diazoxide, doxazosin, enalapril, eposartan, losartan, valsartan, felodipine, fenoldopam, fosinopril, guanabenz, guanadrel, guanethidine, guanfacine, hydralazine, metyrosine, minoxidil, nicardipine, nifedipine, nisoldipine, phenoxybenzamine, prazosin, quinapril, reserpine, and terazosin.

Cardiovascular preparations. Cardiovascular preparations include, by way of example, angiotensin converting enzyme (ACE) inhibitors such as enalapril, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-benzazepine-1-acetic acid or 3-(1-ethoxycarbonyl-3-phenyl-(1S)-propylamino)-2,3,4,5-tetrahydro-2-oxo-(3S)-benzazepine-1-acetic acid monohydrochloride; cardiac glycosides such as digoxin and digitoxin; inotropes such as amrinone and milrinone; calcium channel blockers such as verapamil, nifedipine, nicardipene, felodipine, isradipine, nimodipine, bepridil, amlodipine and diltiazem; beta-blockers such as atenolol, metoprolol; pindolol, propafenone, propranolol, esmolol, sotalol, timolol, and acebutolol; antiarrhythmics such as moricizine, ibutilide, procainamide, quinidine, disopyramide, lidocaine, phenytoin, tocainide, mexiletine, flecainide, encainide, bretylium and amiodarone; and cardioprotective agents such as dexrazoxane and leucovorin; and vasodilators such as nitroglycerin; and diuretic agents such as hydrochlorothiazide, furosemide, bumetanide, ethacrynic acid, torsemide, azosemide, muzolimine, piretanide, and tripamide.

Anti-viral agents. Antiviral agents that can be delivered using the present dosage forms include the antiherpes agents acyclovir, famciclovir, foscarnet, ganciclovir, idoxuridine, sorivudine, trifluridine, valacyclovir, and vidarabine; the antiretroviral agents didanosine, stavudine, zalcitabine, and zidovudine; and other antiviral agents such as amantadine, interferon alpha, ribavirin and rimantadine.

Sex steroids. The sex steroids include, first of all, progestogens such as acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. Androgenic agents, also included within the general class of sex steroids, are drugs such as the naturally occurring androgens androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, 4-dihydrotestosterone (DHT; also termed “stanolone”), 5α-dihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone.

Muscarinic receptor agonists and antagonists. Muscarinic receptor agonists include, by way of example: choline esters such as acetylcholine, methacholine, carbachol, bethanechol (carbamylmethylcholine), bethanechol chloride, cholinomimetic natural alkaloids and synthetic analogs thereof, including pilocarpine, muscarine, McN-A-343, and oxotremorine. Muscarinic receptor antagonists are generally belladonna alkaloids or semisynthetic or synthetic analogs thereof, such as atropine, scopolamine, homatropine, homatropine methyl bromide, ipratropium, methantheline, methscopolamine and tiotropium.

Peptide drugs. Peptidyl drugs include the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP),and vasopressin. Other peptidyl drugs are the cytokines, e.g., colony stimulating factor 4, heparin binding neurotrophic factor (HBNF), interferon-α, interferon α-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-α, granuloycte colony-stimulating factor (G-CSF), granulocyte-macrophage colony-stimulating factor (GM-CSF), macrophage colony-stimulating factor, midkine (MD), and thymopoietin. Still other peptidyl drugs that can be advantageously delivered using the present systems include endorphins (e.g., dermorphin, dynorphin, α-endorphin, β-endorphin, γ-endorphin, σ-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof

Genetic material may also be delivered using the present dosage forms, e.g., nucleic acids, RNA, DNA, recombinant RNA, recombinant DNA, antisense RNA, antisense DNA, ribozymes, ribooligonucleotides, deoxyribonucleotides, antisense ribooligonucleotides, and antisense deoxyribooligonucleotides. Representative genes include those encoding for vascular endothelial growth factor, fibroblast growth factor, Bcl-2, cystic fibrosis transmembrane regulator, nerve growth factor, human growth factor, erythropoietin, tumor necrosis factor, and interleukin-2, as well as histocompatibility genes such as HLA-B7.

In contrast to many erodible dosage forms, the low variability of the present dosage forms is particularly important for poorly soluble drugs such as phenytoin and carbamazepine, both anticonvulsant drugs used in the treatment of epilepsy, as noted above, and for which, due to wide variation in drug absorption from patient to patient, doctors must now titrate their patients individually to find a proper (i.e., safe and effective) dosage regimen. In this regard, the dosage forms of the invention are useful for more consistent delivery of sparingly soluble drugs that have a narrow therapeutic index, i.e., drugs for which the toxic dose is not significantly higher than the effective dose.

The dosage forms of the present invention are particularly useful for delivering drugs directly into the stomach for an extended period of time, for example, when the drug is preferentially absorbed in the small intestine (e.g., ciprofloxacin), or for providing continuous, local-only (non-systemic) action, for example, when the drug is calcium carbonate, and which when incorporated into the dosage forms of the present invention becomes a non-systemic, controlled-release antacid. The dosage forms are also useful for delivering drugs continuously to the stomach that are only soluble in that portion of the gastrointestinal tract. For instance, the dosage forms of the present invention are useful for the delivery of calcium carbonate or other calcium salts intended to be used as an antacid or as a dietary supplement to prevent osteoporosis. Calcium salts are soluble in the stomach but not in the remainder of the G.I. tract, as a result of the presence of stomach acid. With conventional dosage forms, the dwell time of the delivered agent in the stomach is limited usually to only about 20 to 40 minutes, which, in turn, results in a calcium availability of only about 15 to 30%. As a consequence, extremely large dosage forms (2.5 grams), which are difficult for patients to swallow, are commonly utilized. In contrast, by providing controlled delivery for about 4 to 8 hours, plus gastric retention of from about 4 to 8 hours, the dosage forms of the present invention assure more complete bioavailability of elemental calcium from the administered drug, i.e., calcium carbonate. This results in a greater likelihood of patients receiving the intended dose and, also, avoids the need for impractically large dosage forms.

The dosage forms of the present invention are also useful for delivering drugs to treat local disorders of the stomach, such as those that are effective for eradicating Helicobacter pylori (H. pylori) from the submucosal tissue of the stomach, to treat stomach and duodenal ulcers, to treat gastritis and esophagitis and to reduce risk of gastric carcinoma. The dosage forms of the present invention are particularly useful for the foregoing indications because they provide enhanced gastric retention and prolonged release. In a preferred such embodiment, a dosage form of the invention will comprise a combination of (a) bismuth (e.g., as bismuth subsalicylate), (b) an antibiotic such as tetracycline, amoxicillin, thiamphenicol, or clarithromycin, and (c) a proton pump inhibitor, such as omeprazole. A combination of bismuth subsalicylate, thiamphenicol and omeprazole is a particularly preferred combination that may be delivered using the dosage forms of the present invention for the eradication of H. pylori.

Drugs delivered from the gastric-retentive, controlled delivery dosage forms of the invention continuously bathe the stomach and upper part of the small intestine—in particular, the duodenum—for many hours. These sites, particularly the upper region of the small intestine, are the sites of most efficient absorption for many drugs. By continually supplying the drug to its most efficient site of absorption, the dosage forms of the present invention allow for more effective oral use of many drugs.

Since the dosage forms of the present invention provide the drug by means of a continuous delivery instead of the pulse-entry delivery associated with conventional dosage forms, two particularly significant benefits result from their use: (1) a reduction in side effects from the drug(s); and (2) an ability to effect treatment with less frequent administration of the drug(s) being used. For instance, when administered in a conventional dosage form, the sparingly soluble drug, ciprofloxacin, an antibiotic administered to treat bacterial infections such as urinary tract infections, is currently given two times daily and may be frequently accompanied by gastrointestinal side effects such as diarrhea. However, using the dosage forms of the present invention, the number of daily doses can be decreased to one with a lower incidence of side effects.

The invention is not, however, limited to dosage forms for delivering poorly soluble drugs. Drugs having moderate to substantial aqueous solubility can also be delivered using the present dosage forms. If necessary, they may or may not be encased in a protective vesicle and/or coated with a delayed release (e.g., enteric) coating so that a controlled release profile is maintained. Preferred such drugs include, without limitation, metformin hydrochloride, vancomycin hydrochloride, captopril, enalopril or its salts, erythromycin lactobionate, ranitidine hydrochloride, sertraline hydrochloride, ticlopidine hydrochloride, amoxicillin, cefuroxime axetil, cefaclor, clindamycin, doxifluridine, gabapentin, tramadol, fluoxetine hydrochloride, ciprofloxacin hydrochloride, acyclovir, levodopa, ganciclovir, bupropion, lisinopril, losartan, and esters of ampicillin. Particularly preferred such drugs are metformin hydrochloride, ciprofloxacin hydrochloride, gabapentin, lisinopril, enalopril, losartan, and sertraline hydrochloride.

Any of the aforementioned active agents may also be administered in combination using the present dosage forms. Examples of particularly important drug combination products include, but are not limited to, an ACE inhibitor or an angiotensin II antagonist in combination with a diuretic. Specific examples of ACE inhibitors are captopril, lisinopril, or enalopril, and examples of diuretics include triampterine, furosemide, bumetanide, and hydrochlorothiazide. Alternatively, either of these diuretics can advantageously be used in combination with a beta-adrenergic blocking agent such as propranolol, timolol or metoprolol. These particular combinations are useful in cardiovascular medicine, and provide advantages of reduced cost over separate administrations of the different drugs, plus the particular advantage of reduced side effects and enhanced patient compliance. For example, it has been shown that small doses of a diuretic plus small doses of either an ACE inhibitor or a beta blocker provide the additive effects of lowering blood pressure without the additive side effects of the two together.

The benefits of this invention will be achieved over a wide range of drug loadings, with the weight ratio of drug to polymer generally, although not necessarily, ranging from 1:1000 to about 85:15, typically from 1:500 to about 85:15, more typically from 1:400 to about 80:20. Preferred loadings (expressed in terms of the weight percent of drug relative to total of drug and polymer) are those within the range of approximately 10% to 80%, more preferably within the range of approximately 30% to 80%, and most preferably, in certain cases, within the range of approximately 30% to 70%. For some applications, however, the benefits will be obtained with drug loadings as low as 0.01%, as may be inferred from the aforementioned ratios.

V. Dosage Forms, Protective Vesicles and Coatings:

The formulations of this invention are typically in the form of tablets. Other formulations contain the matrix/active agent particles in capsules or compressed into a tablet. The encapsulating material should be highly soluble so that the particles are freed and rapidly dispersed in the stomach after the capsule is ingested. Such dosage forms are prepared using conventional methods known to those in the field of pharmaceutical formulation and described in the pertinent texts, e.g., in Gennaro, A. R., editor, Remington: The Science and Practice of Pharmacy, cited supra. Tablets and capsules represent the most convenient oral dosage forms, in which cases solid pharmaceutical carriers are employed.

Tablets may be manufactured using standard tablet processing procedures and equipment. One method for forming tablets is by direct compression of a particulate composition, with the individual particles of the composition comprised of a matrix of a biocompatible, hydrophilic, erodible polymer having the active agent incorporated therein, alone or in combination with one or more carriers, additives, or the like. As an alternative to direct compression, tablets can be prepared using wet-granulation or dry-granulation processes. Tablets may also be molded rather than compressed, starting with a moist or otherwise tractable material, and using injection or compression molding techniques using suitable molds fitted to a compression unit. Tablets may also be prepared by extrusion in the form of a paste, into a mold, or to provide an extrudate to be “cut” into tablets. However, compression and granulation techniques are preferred, with direct compression particularly preferred.

Tablets prepared for oral administration according to the invention, and manufactured using direct compression, will generally contain other materials such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate (in a concentration of from 0.25% to 3% by weight, preferably less than 1% by weight), calcium stearate, stearic acid, and hydrogenated vegetable oil (preferably comprised of hydrogenated and refined triglycerides of stearic and palmitic acids at about 1% to 5% by weight, most preferably less than about 2% by weight). Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions.

As noted above, the active agent/polymer matrix particles of the invention may also be administered in packed capsules. Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

As previously mentioned, the dosage forms of the present invention are particularly useful for delivering drugs having little or no solubility in water. However, the dosage forms of the present invention can be used to deliver a drug incorporated into a protective vesicle and/or coated with a protective (e.g., enteric) coating, in which case the drug can be, but is not necessarily, water soluble. That is, as explained in U.S. Pat. No. 5,972,389 to Shell et al., cited supra, water-soluble drugs can be rendered sparingly soluble or insoluble when incorporated into protective vesicles and/or coated with a protective coating. Suitable vesicles include, but are not limited to, liposomes and nanoparticles, e.g., nanospheres, nanocapsules and nanocrystals composed of amino acids.

Certain water-soluble drugs may be incorporated directly into the dosage form without prior incorporation into vesicles. This occurs when the solubility of the drug is less than 25% (w/w) at 20° C. or when the molecular weight of the active compound is greater than 300 daltons.

By incorporating a drug either in a protective vesicle or enteric coating into the dosage form of the present invention, the benefits of gastric retention and gradual release to the G.I. tract are combined with the advantageous properties of the vesicle or enteric coating. Advantageous properties associated with the use of protective vesicles and coatings include, for example, protecting the drug from the detrimental environment of the G.I. tract (e.g., from degradative enzymes and low pH), enhancing drug absorption and/or altering drug solubility. This is particularly true of reducing an insoluble drug to nanoparticles with or without surfactant or polymeric additives and incorporating these nanoparticles into the gastric retentive dosage form. In this context, the drug in combination with either agent is continuously and gradually released from the gastric-retentive system to bathe the duodenum and the remainder of the small intestine in a prolonged manner which is determined by the rate at which the polymer erodes. Moreover, less drug may be required to achieve therapeutic efficacy because less drug may be lost as a result of degradation within the stomach. Once released, the drug stabilized through the use of a vesicle or enteric coating may be more readily available for absorption through the intestine.

In addition, the vesicle employed can be selected to improve the bioavailability of a drug by bypassing the liver and taking the drug directly into the lymphatic system. For example, Peyer's patches are regions lining approximately 25% of the G.I. tract and function as absorption sites to the lymphatic system. Vesicles such as liposomes have been shown to be preferentially taken up by Peyer's patches. By incorporating an antigen-associated liposome into the dosage forms of the present invention, controlled and continuous delivery of the antigen to the lymphoid system over a period of several hours is possible as a result of the preferential absorption of the liposome by the Peyer's patches. Also, the liposome provides further protection of the drug from the time it leaves the dosage form until it reaches the absorption site. By delivering the antigen in this manner, there is no longer a need to ingest large amounts of the antigen to avoid degradative gastric acidity and proteolytic enzymes. Methods for preparing liposome encapsulated drug systems are known to and used by those of skill in the art. A general discussion, which includes an extensive bibliography regarding liposomes and methods for their preparation, can be found in “Liposomes, A Practical Approach,” R.R.C New, Ed., 1990.

Further examples of such vesicles include microparticulate systems, which are exemplified by nanoparticles and proteinoid and amino acid microspheres and pharmacosomes. Nanoparticles include, for example, nanospheres, nanocapsules, and nanocrystals. The matrix-like structure of the nanosphere allows the drug to be contained either within the matrix or coated on the outside. Nanoparticles may also consist of stabilized submicron structures of drug with or without surfactant or polymeric additives. Nanocapsules have a shell of polymeric material and, as with the nanospheres, the drug can be contained either within the shell or coated on the outside. Polymers that can be used to prepare the nanoparticles include, but are not limited to, polyacrylamide, poly(alkyl methacrylates), poly(alkyl cyanoacrylates), polyglutaraldehyde, poly(lactide-co-glycolide) and albumin. For details pertaining to nanoparticle preparation, see, e.g., Allemann, E., et al., “Drug-Loaded Nanoparticles—Preparation Methods and Drug Targeting Issues,” Eur. J. Pharm. Biopharm. 39(5):173-191, 193.

As noted above, when employing protective vesicles, the drug need not be sparingly soluble. Thus, the dosage forms of the invention are applicable to drugs of higher solubility in that the rate at which the drug solubilizes is retarded due to the vesicle as it is bound up with the dosage form. As the dosage form erodes, the vesicle containing the drug is freed to the G.I. tract and allowed to pass into the intestines. As a result, a greater amount of drug is retained in the stomach for a longer period of time when compared to the administration of either drug alone or the drug within the vesicle in the absence of the dosage form.

The drug particles may also be provided with a protective coating to ensure delayed release, i.e., a coating that serves to delay dissolution of the drug particles until they have passed out of the acidic environment of the stomach. This is particularly preferred when the drug is moderately to significantly water-soluble, so as to maintain the desired controlled release profile. Drug particles with delayed release coatings may be manufactured using standard coating procedures and equipment. Such procedures are known to those skilled in the art and described in the pertinent texts, e.g., in Remington, supra. Generally, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof Preferred enteric coatings herein are comprised of methacrylic acid copolymers, types A, B, or C, which are commercially available from Rohm Tech, Inc. (Malden, Mass.), and water-based dispersions of cellulose acetate phthalate latex, which is commercially available from Eastman Fine Chemicals (Kingsport, Tenn.).

The dosage forms of the invention may also be formulated as bilayer tablets, trilayer tablets, or shell-and-core tablets, with bilayer and trilayer tablets preferred. In any of these embodiments wherein a dosage form is composed of two or more discrete regions each with different functions or attributes (e.g., a bilayer tablet with one layer being primarily swellable, and the other layer being primarily erodible), two or more drugs can be delivered in two or more different regions (e.g., layers), where the polymer or polymers in each region are tailored to provide a dissolution, erosion and/or release profile, taking the solubility and molecular weight of the drug into account. For example, a bilayer tablet may be prepared with one drug incorporated into an erosional layer and a second drug, which may or may not be identical to the first drug, incorporated into a swelling layer, or a single drug may be incorporated into an erosional layer, with no active agent in the swelling layer. As another example, a trilayer tablet may be prepared with a two outer layers containing drug, comprised of a polymer that is primarily erodible, with a swellable intermediate layer therebetween. The function of the swelling layer is to provide sufficient particle size throughout the entire period of drug delivery to promote gastric retention in the fed mode. In other embodiments, a drug may be included in a coating for immediate release.

VI. Dosage and Administration:

The dose of drugs from conventional medication forms is specified in terms of drug concentration and administration frequency. In contrast, because the dosage forms of the present invention deliver a drug by continuous, controlled release, a dose of medication used in the disclosed systems is specified by drug release rate and by duration of release. The continuous, controlled delivery feature of the system allows for (a) a reduction in drug side effects, since only the level needed is provided to the patient, and (b) a reduction in the number of doses per day.

Different drugs have different biological half-lives, which determine their required frequency of administration (once daily, four times daily, etc.). Thus, when two or more drugs are co-administered in one conventional medication unit, an unfavorable compromise is often required, resulting in an underdose of one drug and an overdose of the other. One of the advantages of the dosage forms of the present invention is that they can be used to deliver multiple drugs without requiring such compromises. For example, in an alternative embodiment, a plurality of drug-containing, spherical, spheroidal- or cylindrical-shaped particles are provided, some of the particles containing a first drug/polymer composition designed to release the first drug at its ideal rate and duration (dose), while other particles contain a second drug/polymer composition designed to release the second drug at its ideal rate and duration. In this embodiment, the polymers or polymer molecular weight values used for each of the drugs can be the same or different. Control of the release rate of the differing drugs can also be obtained by combining different numbers of each of the drug/polymer particles in a common dosage form such as a capsule. For example, where two drugs are combined in a capsule made from five particles, three particles would contain one drug and the other two particles would contain the other drug.

Furthermore, the invention provides dosage forms of separate particles, each comprising polymers that may erode at different rates. As a result, the dosage forms of the present invention achieve a plurality of drug delivery rates. For example, the dosage form may comprise three particles, the first and second containing a swellable polymer that erodes and delivers drug over a period of 4 hours, and the third containing a swellable polymer that erodes and delivers drug over a period of 8 hours. In this regard, requisite erosion rates can be achieved by combining polymers of differing erosion rates into a single particle.

In addition, the invention provides dosage forms of separate particles, some comprising polymers that swell, but do not erode and some comprising polymers that swell and erode (with either the same or differing erosion rates). As a result, the dosage forms can achieve a plurality of delivery rates. For example, the dosage form may comprise three particles, the first containing a swellable polymer that delivers drug over a period of 8 hours, the second containing a swellable/erodible polymer that erodes and delivers drug over a period of 4 hours, and the third containing a swellable/erodible polymer that erodes and delivers drug over a period of 6 hours. In this example, the dosage form may contain one, two or three different drugs.

Drugs that are otherwise chemically incompatible when formulated together can be delivered simultaneously via separate swellable particles contained in a single dosage form. For example, the incompatibility of aspirin and prednisolone can be overcome with a dosage form comprising a first swellable particle with one drug and a second swellable particle with the other. In this manner, the gastric retention and simultaneous delivery of a great number of different drugs is now possible.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXAMPLE 1

Drug dosage forms containing topiramate, an anti-epileptic drug with a water solubility of 1% at 20° C., were prepared in the form of compressed tablets containing swellable, erodible matrix particles with the active agent therein. The in vitro release profile of the tablets was evaluated using a USP Dissolution Test and a USP Disintegration Test, in order to determine which of the latter two tests provided a better correlation to in vivo results.

The matrix particles in the tablets were formulated so as to contain 20 wt. % Polyox® N-60K poly(ethylene oxide) (number average molecular weight approximately 2,000,000), 58.07 wt. % Polyox® N-80 (number average molecular weight approximately 200,000), and 0.5 wt. % magnesium stearate. The weight of each tablet was 600 mg, tablet hardness was approximately 17.1 kP, and approximate tablet dimensions were 7.2×5.3×18.7 mm. When hydrated under static conditions, the increase in tablet size was found to be approximately 60% within two hours. These tablets were tested in a Distek® 2100B Dissolution System, using the USP Dissolution Test described in USP 24-NF 19, Supplement 4, Section 711, with a paddle speed of 50 rpm in 900 ml of deionized water. The resulting release rate curve showed an almost zero-order release, with 90% of the drug released from the dosage form by eight hours.

The in vivo release profile was determined using visual observation and fluoroscopy in the four beagle dogs, with barium sulfate substituted for topiramate to render the tablet radio-opaque. One tablet was administered to each of the four dogs with a small amount of water approximately 30 minutes after the dogs were fed 50 gm of a standard meal (50:50 wet:dry food). The tablet was observed in the dog's stomach, gradually reducing in size until only very small particles were visible at 1.25 hours. This was consistent for all four dogs.

The tablets were also tested in a USP Disintegration Apparatus (55-mm stroke at 30 strokes/min) with a fluted disk in place. The tablets gradually eroded over time with approximately 5% of the tablet remaining at 2 hours.

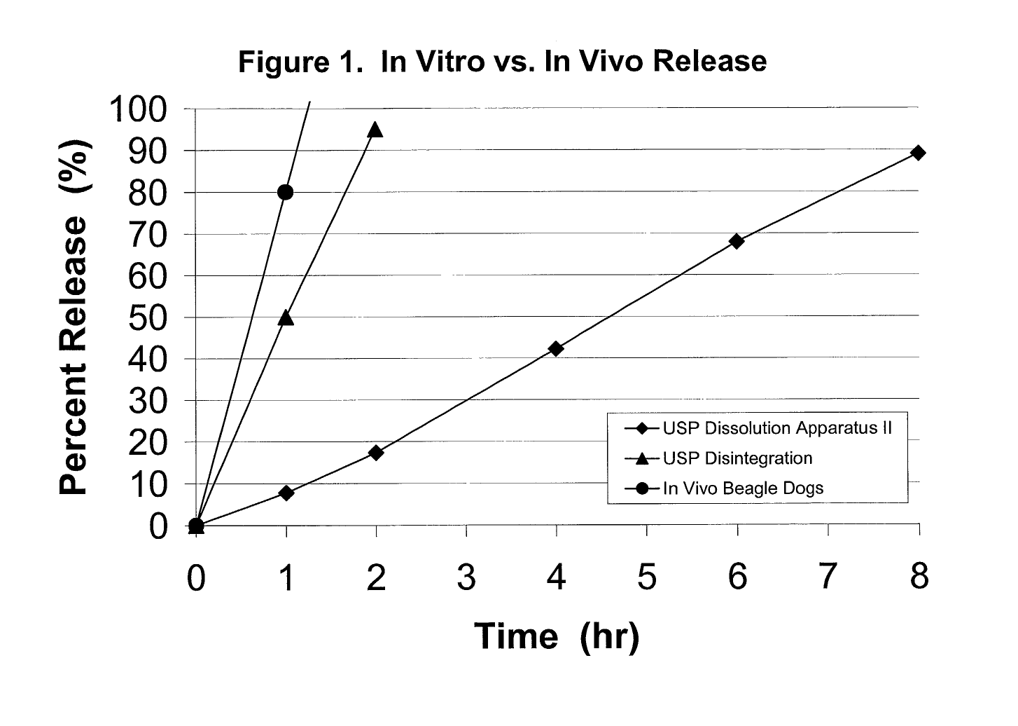

The resulting curves from these three tests are shown in FIG. 1. Additional work has indicated an in vivo/in vitro correlation of 1.6 for topiramate formulations. Data generated from the disintegration testing has indicated that the Polyox® N-80 (200,000 molecular weight) acts more like a disintegrant than a binder. The disintegrating influence of the Polyox® N-80 seems to be independent of the presence of higher molecular weight poly(ethylene oxide)s such as Polyox® N-60K. Although the presence of the higher molecular weight polymers influences the swelling capacity of the matrix, they seem to have little impact as a binder to counteract the disintegration facilitated by the lower molecular weight Polyox® N-80. This was not evident in the release rate profiles obtained from the standard dissolution testing with the USP Dissolution Apparatus II.

To formulate an extended release swellable/erodible tablet based on the release rates obtained from the USP Dissolution Apparatus II would most likely result in unacceptable clinical results. Although the USP Disintegration Apparatus was designed to test immediate release dosage forms, it is a more accurate tool in predicting in vivo erosion of matrix systems. The disintegration apparatus can simulate mechanical action, and the test media can be changed to incorporate some of the other factors acting on the dosage form in vivo—enzyme effects, pH effects, etc.

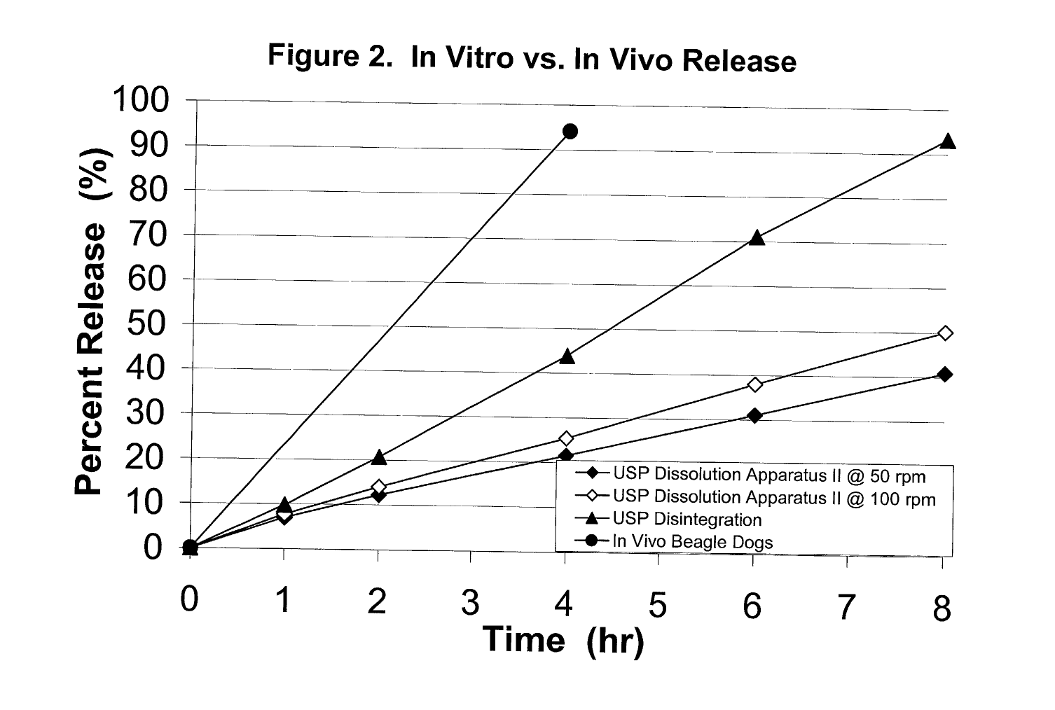

The dog has been determined to be a good model for estimating human retention and gastric transit time. FIG. 2 shows the release profile of a dosage form that was formulated to disintegrate in approximately 4 hours in a dog's stomach. The dosage form disintegrated in approximately 8 hours in a USP Disintegration apparatus, but no disintegration was visible in the USP Dissolution apparatus, even when the paddle speed was increased to 100 rpm. There was, accordingly, a significant difference between the dissolution results and the disintegration results.

This is an indication that for a dosage form wherein drug release is primarily erosion controlled rather than dissolution controlled, the dissolution apparatus should only be used as a quality control tool to characterize the dosage form. Although a correlation would need to be developed for each drug matrix, a far better predictor of in vivo release is the USP Disintegration apparatus.

### EXAMPLE 2

Four batches of barium tablets were manufactured, with each tablet containing: at least one of Polyox® N-60K (as above), Polyox® N-80 (as above), and Polyox® 303 (number average molecular weight 7,000,000); 21.35 wt. % barium sulfate (as a contrast agent), and 0.5 wt. % magnesium stearate (as a lubricant). The tablets were manufactured using direct compression at 3000 lbs. and an automated Carver Press. The polymer content of the dosage forms are identified in Table 1 below:

TABLE 1

| Dosage | | |
|---|---|---|
| Form | Batch # | Polymer/Binder Content |
| GR/1 | 1 | 20.02% Polyox ® N-60K, 58.13% Polyox ® N-80 |
| GR/2 | 2 | 20.02% Polyox ® 303, 21.07% Polyox ® N-80, |
| | | 37.06% microcrystalline cellulose |
| GR/3 | 3 | 50.06% Polyox ® N-60K, 28.09% Polyox ® N-80 |
| GR/4 | 4 | 50.06% Polyox ® N-60K, 28.09% microcrystalline |
| | | cellulose |

Tablet Characterization

The tablets weighed 600 mg each with average modified capsule dimensions of 7.2×4.8×18.6 mm. Tablet characteristics, i.e., weight, height, and hardness, are provided in Table 2.

TABLE 2

| | | | Tablet |
|---|---|---|---|
| Dosage Form | Weight (mg) | Tablet Height (mm) | Hardness (kP) |
| GR/1 | 599.4 ± 0.8 | 4.83 ± 0.03 | 17.8 ± 1.2 |
| GR/2 | 601.2 ± 1.8 | 4.61 ± 0.02 | 20.6 ± 0.9 |
| GR/3 | 600.0 ± 1.1 | 4.84 ± 0.04 | 20.4 ± 1.9 |
| GR/4 | 600.9 ± 1.5 | 4.65 ± 0.01 | 21.3 ± 1.5 |

Swelling Measurements:

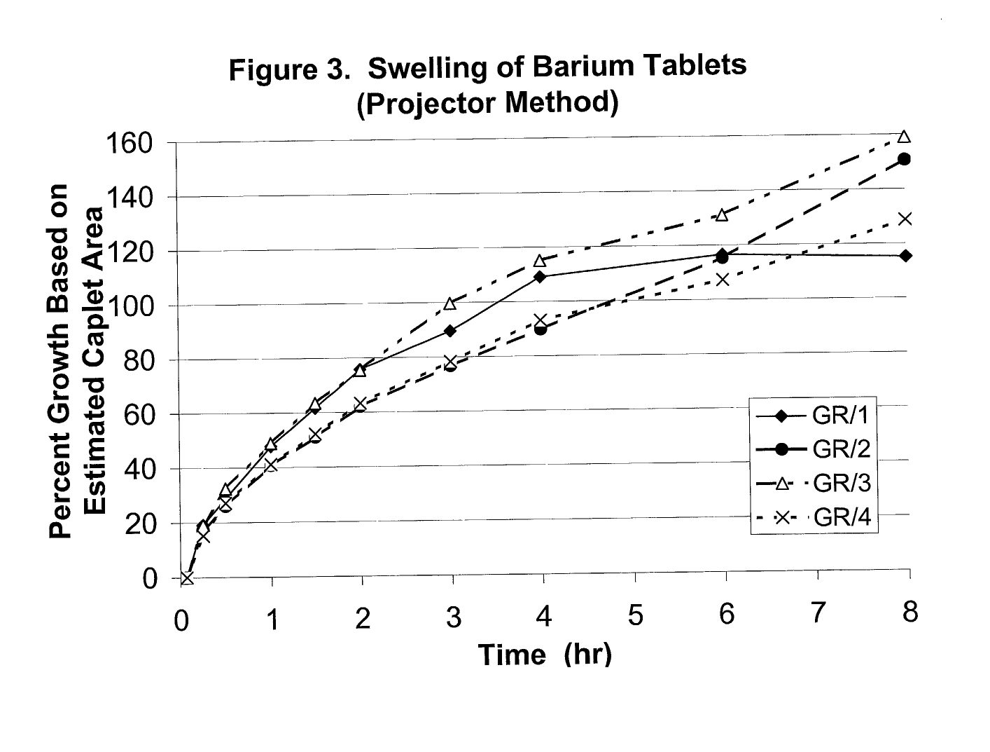

The extent of swelling of these dosage forms was measured by a static projector method. Glass culture dishes pre-partitioned into quadrants were placed on an overhead projector that was positioned approximately two feet from a wall. Three tablets from each batch were placed into a labeled quadrant (one tablet per quadrant) containing enough water to completely submerge the tablets. The image of each tablet was projected onto the wall and the outline of each tablet was traced onto paper. The paper was replaced for each time point: 0, 0.25, 0.5, 1, 2, 3, 4, 6 and 8 hours. The width and length of each projected image was measured and recorded. The extent of swelling was measured by estimating the area of the caplet and comparing the swollen area to the initial area (T=0); see FIG. 3.

The two-dimensional tablet area increased by at least 32% within the first 30 minutes, by at least 50% within the first hour and by at least 72% within the first two hours. The estimated dimensions of the tablets for the first two hours of swelling are provided in Table 3.

TABLE 3

| Dos- | | | |
|---|---|---|---|
| age | Tablet Dimensions | Tablet Dimensions | Tablet Dimensions |
| Form | at T = 0 (mm) | at 1 hour (mm) | at 2 hours (mm) |
| GR/1 | 7.22 × 4.83 × 18.59 | 9.54 × 6.38 × 21.09 | 10.70 × 7.16 × 22.47 |
| GR/2 | 7.26 × 4.61 × 18.68 | 9.35 × 5.94 × 20.70 | 10.22 × 6.49 × 21.89 |
| GR/3 | 7.22 × 4.84 × 18.59 | 9.48 × 6.36 × 21.35 | 10.70 × 7.18 × 22.54 |
| GR/4 | 7.23 × 4.65 × 18.67 | 9.21 × 5.92 × 20.94 | 10.19 × 6.56 × 22.02 |

Disintegration Testing:

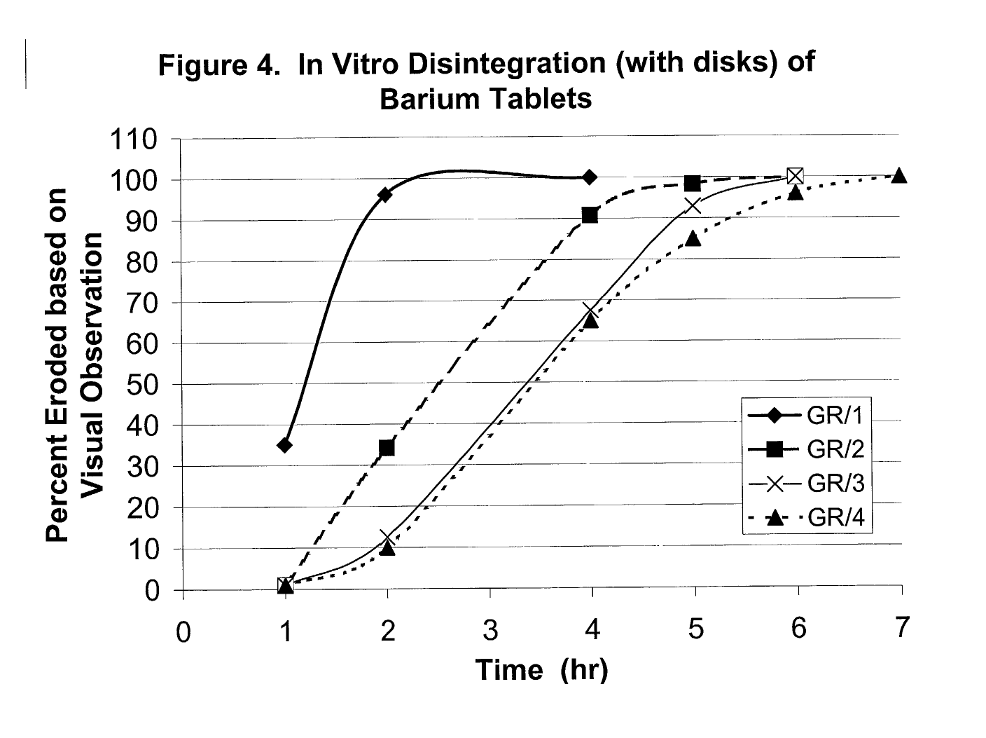

Each of the four GR dosage forms was tested in a USP Disintegration tester with fluted disks (N=3). The results are shown in FIG. 4. The GR/1 dosage form eroded within 2-2.5 hours, the GR/2 within 4-4.5 hours, the GR/3 within 5-6 hours, and the GR/4 within 6-7 hours.

Dog Study Results:

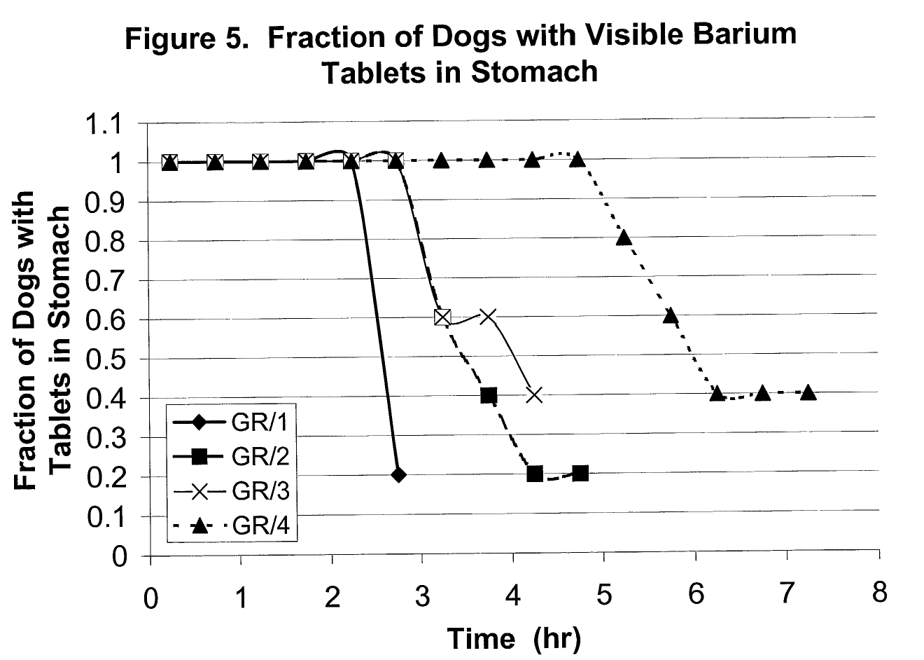

Each of the four dosage forms was administered to each of five beagle dogs with a small amount of water 15 minutes after the dogs were fed 50 gm of their standard meal (50:50 wet:dry food). The dogs were all female, approximately one year old and weighed between 11 and 15 lbs. (5-7 kg). The location of the tablet (in or out of the stomach) and its approximate size was monitored every 30 minutes by fluoroscopy. Table 4 and FIG. 5 summarize the erosion time of the dosage forms in the stomach of the dogs for GR/1, GR/2, GR/3 and GR/4.

TABLE 4

| | Last Time Visualized (hours) | | | |
|---|---|---|---|---|
| Subject # | GR/1 | GR/2 | GR/3 | GR/4 |
| 1 | 2.25 | 3.25 | 3.25 | 5.75 |
| 2 | 2.75+ | 2.75 | 4.75 | 7.25 |
| 3 | 2.25 | 3.75 | 4.75 | 7.25 |
| 4 | 2.25 | 2.75 | 2.75 | 4.75 |
| 5 | 2.25 | 4.75 | 4.25 | 5.25 |
| Mean | 2.35 | 3.45 | 3.95 | 6.05 |
| Std. Dev. | 0.22 | 0.84 | 0.91 | 1.15 |
| Range | 2.25-2.75+ | 2.75-4.75 | 2.75-4.75 | 4.75-7.25 |

For all dosage forms, the tablets can actually be seen decreasing in size over time in the dog's stomachs. The erosion of the dosage forms in the stomach was observed over a two-hour period, with the movement and action of each tablet in the stomach visualized on a monitor prior to recording the image. This allowed the operator to verify that the tablet was not positioned with the end facing the camera and thus presenting a misleading tablet size. There was a good correlation between in vitro disintegration of the various dosage forms and the in vivo erosion in the dogs, as seen in Table 5.

TABLE 5
Comparison of Disintegration Times in vitro
Disintegration Tester vs. in vivo in Dogs

| Dosage Form | in vitro Disintegration (hrs) | in vivo Dog Erosion (hrs) |
|---|---|---|
| GR/1 | 2-2.5 | 2.4 ± 0.2 |
| GR/2 | 4-4.5 | 3.5 ± 0.8 |
| GR/3 | 5-6 | 4.0 ± 0.9 |
| GR/4 | 6-7 | 6.1 ± 1.2 |

We claim:

1. An erodible, gastric-retentive drug dosage form for administering a pharmacologically active agent to the stomach, duodenum, and upper small intestine of a patient, the dosage form comprising the pharmacologically active agent incorporated in a matrix of at least one biocompatible, hydrophilic polymer that (a) swells in the presence of water in gastric fluid such that the size of the dosage form is sufficiently increased to provide gastric retention in the stomach of a patient in whom the fed mode has been induced, (b) gradually erodes within the gastrointestinal tract over a determinable time period, and (c) releases the active agent throughout the determinable time period, wherein the dosage form is formulated so as to provide an active agent release profile in vivo that corresponds to a desired active agent release profile obtained for the dosage form in vitro using USP disintegration test equipment.

2. The dosage form of claim 1, wherein a first fraction of the active agent is released from the dosage form by diffusing out of the polymer matrix as a result of (a) and a second fraction of the active agent is released from the dosage form by erosion of the polymer matrix during (b).

3. The dosage form of claim 2, wherein the second fraction is greater than the first fraction.

4. The dosage form of claim 3, wherein at least 75 wt. % of the active agent is released within the determinable time period.

5. The dosage form of claim 4, wherein at least 85 wt. % of the active agent is released within the determinable time period.

6. The dosage form of claim 1, wherein the at least one biocompatible hydrophilic polymer is selected from the group consisting of: polyalkylene oxides; cellulosic polymers; acrylic acid and methacrylic acid polymers, and esters thereof; maleic anhydride polymers; polymaleic acid; poly(acrylamides); poly(olefinic alcohol)s; poly(N-vinyl lactams); polyols; polyoxyethylated saccharides; polyoxazolines; polyvinylamines; polyvinylacetates; polyimines; starch and starch-based polymers; polyurethane hydrogels; chitosan; polysaccharide gums; zein; shellac-based polymers; and copolymers and mixtures thereof

7. The dosage form of claim 6, wherein the at least one biocompatible hydrophilic polymer is a polyalkylene oxide polymer or copolymer, a cellulosic polymer, a gum, or a mixture thereof.

8. The dosage form of claim 7, wherein the at least one biocompatible hydrophilic polymer is a polyalkylene oxide selected from the group consisting of poly(ethylene oxide), poly(ethylene oxide-co-propylene oxide), and mixtures thereof.

9. The dosage form of claim 8, wherein the at least one biocompatible hydrophilic polymer is poly(ethylene oxide) optionally in admixture with poly(ethylene oxide-co-propylene oxide).

10. The dosage form of claim 6, wherein the at least one biocompatible hydrophilic polymer is a cellulosic polymer selected from the group consisting of hydroxymethylcellulose, hydroxyethylcellulose, hydroxypropylcellulose, hydroxypropyl methylcellulose, carboxymethylcellulose, and mixtures thereof.

11. The dosage form of claim 6, wherein the at least one biocompatible hydrophilic polymer is xanthan gum.

12. The dosage form of claim 1, wherein the at least one biocompatible hydrophilic polymer has a number average molecular weight in the range of approximately 5,000 and 20,000,000.

13. The dosage form of claim 1, wherein the weight ratio of the active agent to the biocompatible hydrophilic polymer is in the range of about 1:500 to about 85:15.

14. The dosage form of claim 13, wherein the weight ratio of the active agent to the biocompatible hydrophilic polymer is in the range of about 5:95 to about 80:20.

15. The dosage form of claim 14, wherein the weight ratio of the active agent to the biocompatible hydrophilic polymer is in the range of about 30:70 to about 80:20.

16. The dosage form of claim 15, wherein the weight ratio of the active agent to the biocompatible hydrophilic polymer is in the range of about 30:70 to about 70:30.

17. The dosage form of claim 1, wherein at least one of the biocompatible hydrophilic polymers is crosslinked.

18. The dosage form of claim 1, wherein the active agent has an aqueous solubility of less than about 25 wt. % at 20° C.

19. The dosage form of claim 18, wherein the active agent has an aqueous solubility of less than about 10 wt. % at 20° C.

20. The dosage form of claim 19, wherein the active agent has an aqueous solubility of less than about 5 wt. % at 20° C.

21. The dosage form of claim 1, wherein the active agent has a molecular weight greater than 300 daltons.

22. The dosage form of claim 18, wherein the at least one biocompatible hydrophilic polymer has a number average molecular weight in the range of about 10,000 to 8,000,000.

23. The dosage form of claim 18, wherein the active agent is selected from the group consisting of topiramate, nifedipine, acyclovir, alprazolam, phenytoin, carbamazepine, ranitidine, cimetidine, famotidine, clozapine, nizatidine, omeprazole, gemfibrozil, lovastatin, nitrofurantoin, losartan, docetaxel and paclitaxel.

24. The dosage form of claim 23, wherein the active agent is topiramate.

25. The dosage form of claim 23, wherein the active agent is paclitaxel.

26. The dosage form of claim 18, wherein the active agent is a Helicobacter pylori eradicant.

27. The dosage form of claim 26, wherein said eradicant is selected from the group consisting of bismuth subsalicylate, bismuth citrate, amoxicillin, tetracycline, minocycline, doxycycline, clarithromycin, thiamphenicol, metronidazole, omeprazole, ranitidine, cimetidine, famotidine and combinations thereof.

28. The dosage form of claim 27, wherein said eradicant is bismuth subsalicylate.

29. The dosage form of claim 1, wherein the active agent is contained within a vesicle.

30. The dosage form of claim 29, wherein the active agent is water soluble but rendered sparingly water soluble by the vesicle.

31. The dosage form of claim 30, wherein the vesicle is selected from the group consisting of liposomes, nanoparticles, proteinoid and amino acid microspheres, and pharmacosomes.

32. The dosage form of claim 31, wherein the vesicle is comprised of a nanoparticle.

33. The dosage form of claim 32, wherein the nanoparticle is a nanosphere, a nanocrystal, or a nanocapsule.

34. The dosage form of claim 30, wherein the active agent is selected from the group consisting of metformin hydrochloride, vancomycin hydrochloride, captopril, erythromycin lactobionate, ranitidine hydrochloride, sertraline hydrochloride, ticlopidine hydrochloride, amoxicillin, cefuroxime axetil, cefaclor, clindamycin, doxifluridine, tramadol, fluoxetine hydrochloride, ciprofloxacin hydrochloride, ganciclovir, bupropion, lisinopril, minocycline, doxycycline, and esters of ampicillin.

35. The dosage form of claim 34, wherein the active agent is metformin hydrochloride.

36. The dosage form of claim 34, wherein the active agent is ciprofloxacin hydrochloride.

37. The dosage form of claim 1, wherein the active agent is enterically coated.

38. The dosage form of claim 37, wherein the active agent is water soluble but rendered sparingly water soluble by said vesicle.

39. The dosage form of claim 1, wherein the dosage form is comprised of a tablet.

40. The dosage form of claim 1, wherein the dosage form is comprised of a capsule.

41. A method for selecting an optimized controlled release dosage form for administration to a patient such that the dosage form will have a predetermined drug release profile in vivo, the method comprising:
(a) preparing a plurality of different candidate dosage forms each comprised of a biocompatible, hydrophilic polymer and a pharmacologically active agent incorporated therein;
(b) obtaining the in vitro drug release profile for each candidate dosage form in an aqueous medium in a USP disintegration tester;
(c) comparing the in vitro drug release profiles obtained in (b), and determining which of the in vitro drug release profiles correlates most closely with a desired in vivo drug release profile; and
(d) selecting the dosage form having the determined in vitro drug release profile for administration to a patient.

42. The method of claim 41, wherein the candidate dosage forms are all comprised of the same biocompatible, hydrophilic polymer but differ with respect to the amount or molecular weight thereof.

43. The method of claim 41, wherein the candidate dosage forms all contain the same pharmacologically active agent but differ with respect to the amount thereof.

44. A method for delaying the passage of a pharmacologically active agent through the gastrointestinal tract of a patient, said method comprising orally administering the dosage form of claim 1 to the patient.
